(19) United States
(12) Patent Application Publication
(10) Pub. No.: US 2018/0171280 A1
(43) Pub. Date: Jun. 21, 2018

(54) CELL CULTURE EXPOSURE SYSTEM (CCES)

(71) Applicant: GOVERNMENT OF THE UNITED STATES AS REPRESENTED BY THE ADMINISTRATOR OF THE U.S. ENVIRONMENTAL PROTEC, Washington, DC (US)

(72) Inventors: Mark HIGUCHI, Chapel Hill, NC (US); Jose ZAVALA-MENDEZ, Durham, NC (US); Earl Puckett, Durham, NC (US)

(73) Assignee: GOVERNMENT OF THE UNITED STATES AS REPRESENTED BY THE ADMINISTRATOR OF THE U.S. ENVIRONMENTAL PROTEC, Washington, DC (US)

(21) Appl. No.: 15/821,096

(22) Filed: Nov. 22, 2017

Related U.S. Application Data
(60) Provisional application No. 62/497,565, filed on Nov. 25, 2016.

(51) Int. Cl. C12M 1/00; C12N 5/00; C12M 1/12; C12N 5/071; G01N 33/483; B01L 1/02

(52) U.S. Cl. CPC C12M 41/14; C12N 5/0068; C12M 25/02; C12N 2533/54; G01N 33/4833; B01L 1/025; C12N 5/0688

(57) ABSTRACT

An vitro air-liquid interface (ALI) exposure of cultured cells makes it possible to examine the toxicological properties of the tested air. A method for evaluating effect of a polluting air stream comprises the steps of exposing cells to a membrane until adhesion of the cells has occurred, feeding cells periodically until there is a confluent monolayer of cells on said membranes, aspirating off non-adherent cells, applying fresh media, exposing the cells to over-head stream containing pollutants, removing the cells from the membranes that have been exposed to the over-head stream, then measuring the cells' response to toxins in the over-head stream.

### BACKGROUND OF THE INVENTION

It is well known that air pollution contributes to disease and reduced mortality, yet complete elimination of air pollution is not obtainable at this time. Effective risk management in regulating air pollution requires reliable information regarding the relative toxicological activity from various sources, improved means of evaluating toxicological activity and hazard in complex aerosols (e.g., urban air, combustion emissions, etc.), and better understanding of the biological mechanisms underlying epidemiologically-observed health hazards. Unfortunately, routine experimental analyses of complex aerosols to evaluate source materials, investigate determinants of hazard, and delineate biological mechanisms underlying adverse effects are hampered by the lack of systems for controlled exposure and toxicological analyses of complex aerosols.

A range of in vivo and in vitro assessment tools have been employed to experimentally assess the toxicological effects of aerosols, vapors, and gases (e.g., diluted vehicular emissions and ambient outdoor air). Although in vivo inhalation studies permit assessment of toxicological effects, in vivo studies are costly and time consuming. In addition, the relevance of rodent studies for human risk assessment is disputed. In vitro tools offer potentially more expeditious and affordable alternatives. In vitro systems that expose living cells to the aerosols, vapors or gases in question are needed.

Assessments of multi-pollutant atmospheres are challenging with traditional in vitro tools, including with ALI. For example, most in vitro air pollutant studies have examined particulate matter (PM) suspended in liquid medium. Although such studies have provided insight regarding PM-induced effects, the methods cannot be readily employed to assess the toxicological effects of multi-pollutant aerosols. In addition, to be most realistic, the route of exposure approximate inhalation, which is challenging for an in vitro study.

At present, the main in vitro cell exposure system products in the market are VitroCell® and Cultex®. Both of these cell exposure systems lack high repeatability between similar experiments, are complex to set up and operate, and lack high correlation to in vivo inhalation exposures.

Testing of volatile chemicals in breathing environments (e.g., in relation to the Toxic Substances Control Act) presents additional challenges. Current automated dosing robotics work with liquids or dissolved solids but do not work for volatile chemicals.

### Object of the Invention

It is the purpose of this invention to provide means for in vitro air-liquid interface (ALI) exposures of cultured cells to examine the toxicological properties of the tested air. The inventive means using ALI exposure method uses cells grown on porous membrane inserts (commercially available), which are then inserted into test modules (cell culture plates), for exposure to test aerosols from above (i.e., at the ALI). Cells are subsequently collected to assess toxicological responses that are relevant to human health (e.g., oxidative stress, inflammatory reactions, and genetic damage).

The inventive method of the invention provides a method for reliably assessing the toxicological effects of multi-pollutant aerosols in a manner that is less costly and time-consuming than traditional in vivo studies. The method of the invention provides an in vitro cell exposure system that results in an inhalation-like exposure and can be used with volatile chemicals.

### SUMMARY OF THE INVENTION

The Cell Culture Exposure System (CCES) is designed to more effectively replicate in in vitro conditions and the effect of pollutants on cells, whereby cells are exposed to gases and particulates in an ALI system. The practice of the invention involves growing cells on semi-permeable membranes with basal culture medium, and subsequent ALI aerosol exposure of the apical cell surface from above. The CCES method allows for the use of similar generation and exposure systems without need for in-vivo inhalation exposures. The method of the invention uses in-vitro exposures with minimal conversion and therefore provides means for direct comparisons of effect of various pollutant chemicals on exposed cells.

The method of the invention consists essentially comprises exposing cells to a membrane, (a preferred membrane being a collagen-coated membrane such as exemplified herein) until adhesion of the cells has occurred, feeding cells periodically until there is a confluent monolayer of cells on said membranes, aspirating off non-adherent cells, applying fresh media, exposing the cells to over-head an stream containing pollutants, then removing the cells from the membranes that have been exposed to the over-head stream, followed by measuring the cells for response to toxins in the over-head stream.

### BRIEF DESCRIPTION OF THE DRAWINGS

FIG. 1 is an exploded view of a cell culture exposure system per the invention.

FIG. 2A is a schematic diagram of a cell culture exposure system used to assess gases, per the invention.

FIG. 2B is a schematic diagram of a cell culture exposure system used to assess aerosols, per the invention.

FIG. 3 is a schematic diagram of a cell culture exposure system per an alternative embodiment of the invention.

FIG. 4A is a schematic diagram of CCES bottom plate—side view.

FIG. 4B is a schematic diagram of CCES bottom plate—top view.

FIG. 5 is a schematic diagram of CCES ¼″ baffle plate—top view.

FIG. 6A is a schematic diagram of CCES 24-hole top plate—side view used to assess gases, per the invention.

FIG. 6B is a schematic diagram of CCES 24-hole top plate—top view used to assess gases, per the invention.

FIG. 7A is a schematic diagram of CCES 6-hole top plate—side view used to assess aerosols, per the invention.

FIG. 7B is a schematic diagram of CCES 6-hole top plate—top view used to assess aerosols, per the invention.

FIG. 8 is a schematic diagram of CCES 24-hole plate nozzles used to assess gases, per the invention.

FIG. 9 is a schematic diagram of CCES 6-hole plate nozzles used to assess aerosols, per the invention.

FIG. 10 is a graph showing CellTiter-Glow viability for human primary lung cell exposures to 1,3-Butadiene.

FIG. 11 is a graph showing CellTiter-Glow viability for BEAS-2B exposures to 1,3-Butadiene.

### DETAILED DESCRIPTION

The method of the invention consists essentially of growing cells in a culture, of exposing cells to a membrane, (a preferred membrane being a collagen-coated membrane such as exemplified herein) until adhesion of the cells has occurred, then feeding cells periodically until there is a confluent monolayer of cells on said membranes, aspirating off non-adherent cells, applying fresh media, exposing the cells to over-head an stream containing pollutants, then removing the cells from the membranes that have been exposed to the over-head stream, followed by assessing the cells for response to toxins in the over-head stream.

In the following example, after the adhesion period and aspiration of non-adherent cells 750 μl of fresh KGM was added to each apical compartment. (It is best to use cells within 48-72 h for experiments; otherwise, the cells may become too confluent and no longer form a monolayer, which is required for ALI exposures.) (Suggested Cell Seeding Densities for membranes are related to size Growth Area (cm²)). Seeding Density/well Apical Volume (ml) Basolateral Volume (ml) 6.5 mm (24 well), 0.33 15-30,000 0.1 0.4 24 mm (6 well). Feed cells every 48 h until confluent, at which point the experiment testing should then be performed. After exposure, remove cells by adding 50 μl of trypsin-EDTA to each membrane, allow to sit for ˜8 min, pipette gently and remove cells to an Eppendorf vial of appropriate size for the desired assay.

Preparation of the cells for use in the process of the invention will vary depending on circumstances and the particular cells under study. The following example is for use with BEAS-B2 cells. The particulars disclosed herein were used in the examples and are illustrative of methods for use in practicing the invention.

### Example I: (Using BEAS-B2, S6 Cells)

This protocol is intended for putting BEAS-2B cells at air-liquid-interface (ALI).

### Equipment and Supplies

1) BEAS-2B, S6 cells in culture, 2) KGM-Gold BulletKit were obtained (Lonza Catalog #: 192060), 3) trypsin, 0.25% with EDTA (any brand), 4) Soybean trypsin inhibitor (SBTI; Type II crude powder) (Sigma Catalog # T9128) prepared at 1 mg/ml in DPBS (GIBCO Catalog #14190-144) and sterile filtered through 0.22-μm filters (any brand), 5) 24-well tissue culture plates (Corning), 6) Transwell membranes, Transwell-Clear, or Transwell-COL (CoStar, Cambridge Mass.), (polycarbonate is much cheaper; or one may purchase collagen coated, which are more expensive; either will do). (Transwell filters or membranes are available in a number of sizes and varieties. Check the COSTAR catalog for the appropriate ordering information.), 7) Laminar flow hood (any brand), 8) Sterile plastic pipettes (1, 5, 10, 25 ml; any brand), and a pipette-aid, 9) tissue Culture Incubator 37° C. and 5% CO₂ (any brand), 10) inverted microscope, any brand, with 4, 10, and 20× objectives, 11) collagen (Advanced Biomatrix, Cat #5005-100 ml), 12) high-purity water for molecular biology (any brand), 13) balance that reads to at least 0.1 mg and associated supplies, and 14) sterile, 15-ml polypropylene centrifuge tubes, any brand.

BEAS-2B cells are an immortalized cell type that accumulates mutations over time in culture. Their response to air-liquid interface exposures is somewhat different than the response of primary human bronchial epithelial cells (McCullough et al. 2014). The membranes should be pre-coated with collagen, and this can be done by ether purchasing pre-coated membranes from the manufacturer or by doing it in-house as described below. For the practice of the invention as exemplified, the age of the plates is critical. When reading the lot number of Corning products, the 4th and 5th digits represent the year of manufacture. Using plates older than 2 years' results in poor adherence. The following protocol was followed:

Coating membranes with collagen: Prepare collagen stock solution at 3.1 mg collagen/ml of water. A stock of 100 ml can be prepared and stored at 4° C. for up 2 years. The stock solution was diluted for coating the filters. This was done by adding 377 μl of the stock solution to 14.6 ml of cell-grade sterile water in a sterile tube. This gives ˜15 ml of diluted solution, which is sufficient to coat 12 of the 24-well plates. To each plate was added 50 μl of the diluted solution. The membranes were allowed to dry partially covered overnight in the laminar-flow hood. The next day, the remaining solution was aspirated and each well each well was rinsed with 100 μl of cell-grade water. The inserts were allowed to dry for an hour in the hood followed by plating cells immediately. If cells are not used immediately, place the plates containing the coated membranes into an empty (sterile) T75-flask bag and store at 4° C. (These can be stored for 1 month; but if not used by then, discard.)

Plating cells: Remove cells from a T75 flask by adding 4 ml of trypsin-EDTA, let sit 5-8 min, gently tap the flask when 80-90% of the cells are detached, and then add 1 ml of SBT1. Gently pipette the mixture and rinse the flask bottom with the mixture and then transfer the mixture to a sterile 15-ml polypropylene centrifuge tube. Add 10 ml of KGM; cells from individual flasks can be combined into the tube. Centrifuge cells at 800 rpm (or 125×g) for 5 min at room temperature. Aspirate the supernatant and add 5 ml of 37° C. KGM and triturate the cells to create a single-cell suspension. Determine cell density using a cellometer OP and adjust cell density to the OP. Desired density was obtained (see below) by either adding more media and/or centrifuging and re-suspending in less media. For 24-wells, add 1.5 ml of KGM to the basolateral compartment of each well, and add 2-5×105 cells/r insert in 1 ml of medium to the apical compartment of a collagen-coated membranes. Let cells adhere 16-24 h in the CO2 incubator. After the adhesion period, aspirate off non-adherent cells and then add 750 μl of fresh KGM to each apical compartment. Use cells within 48-72 h for experiments; otherwise, the cells may become too confluent and no longer form a monolayer, which is required for ALI exposures. (Other membrane sizes and seeding densities are shown below. Suggested Cell Seeding Densities for membranes are related to size Growth Area (cm²) Seeding Density/well Apical Volume (ml) Basolateral Volume (ml) 6.5 mm (24 well), 0.33 15-30,000 0.1 0.4 24 mm (6 well). Feed cells every 48 h until confluent, at which point the testing should then be performed. After exposure, cells were removed by adding 50 μl of trypsin-EDTA to each membrane. After ˜8 min, pipette gently and remove cells to an Eppendorf vial of appropriate size for the desired assay.

If cells become overgrown on membranes, such cells should not be used for ALI exposures. It is preferable to dispose of all media and cultures if cell cultures become contaminated. Incubator should be monitored daily for temperature and CO₂ levels. Temperature should be monitored by a thermometer placed inside the incubator, and CO₂ by the gauge on the incubator. The acceptable range of temperature is 35-38° C., and for the CO₂ 3.5 to 6.

Regarding equipment for testing in accord with the examples is shown in the figures.

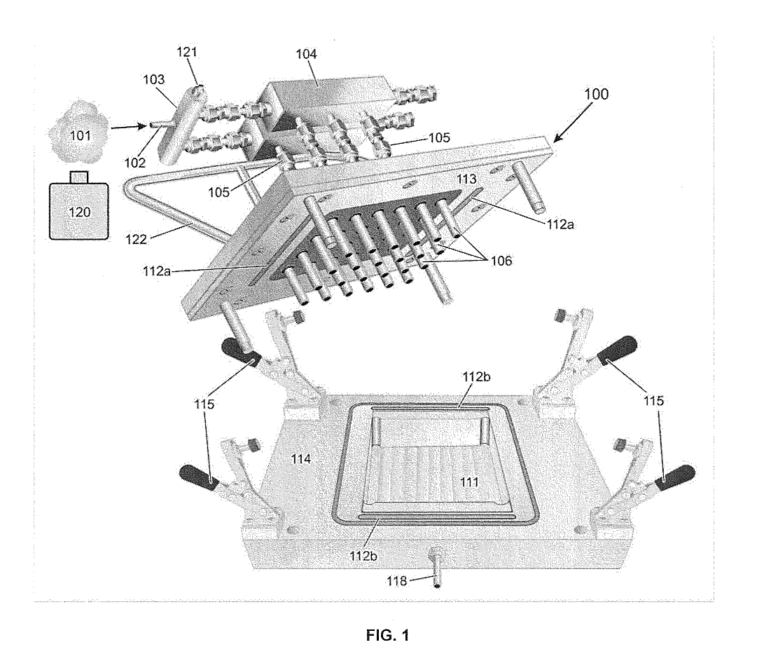

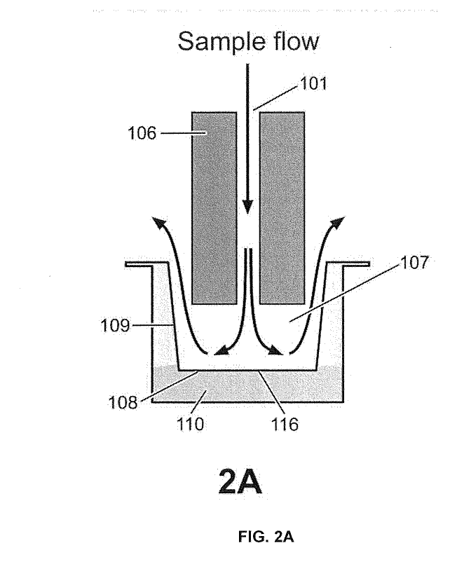

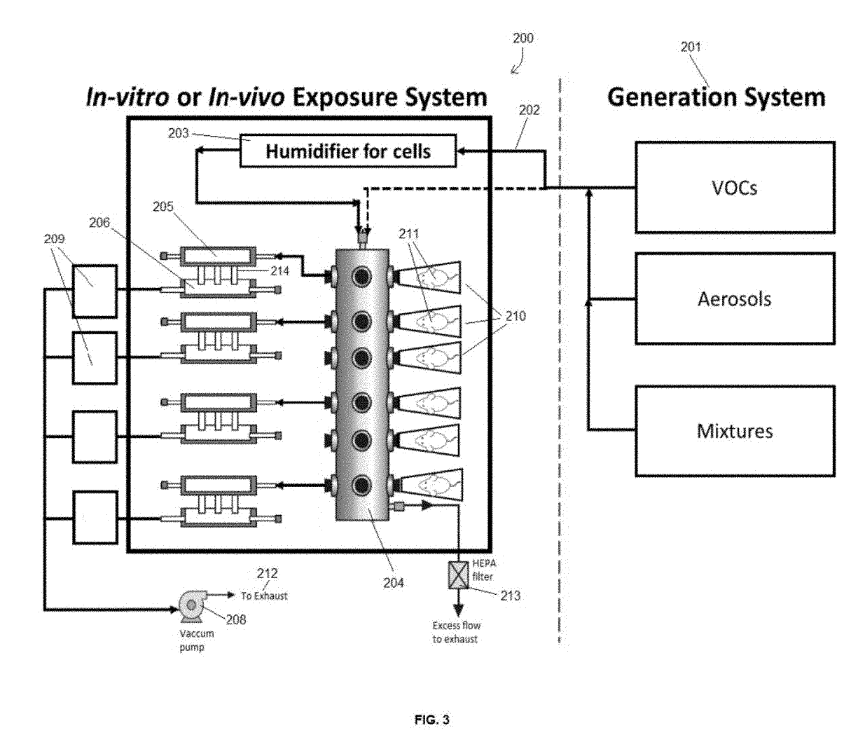

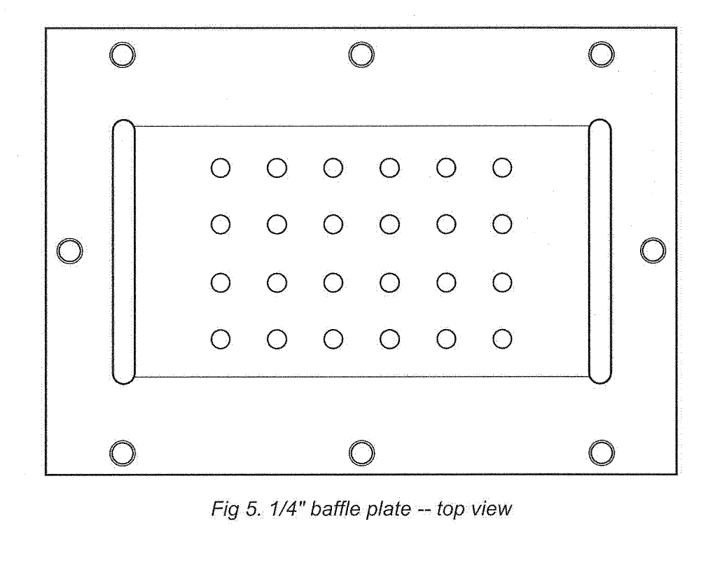

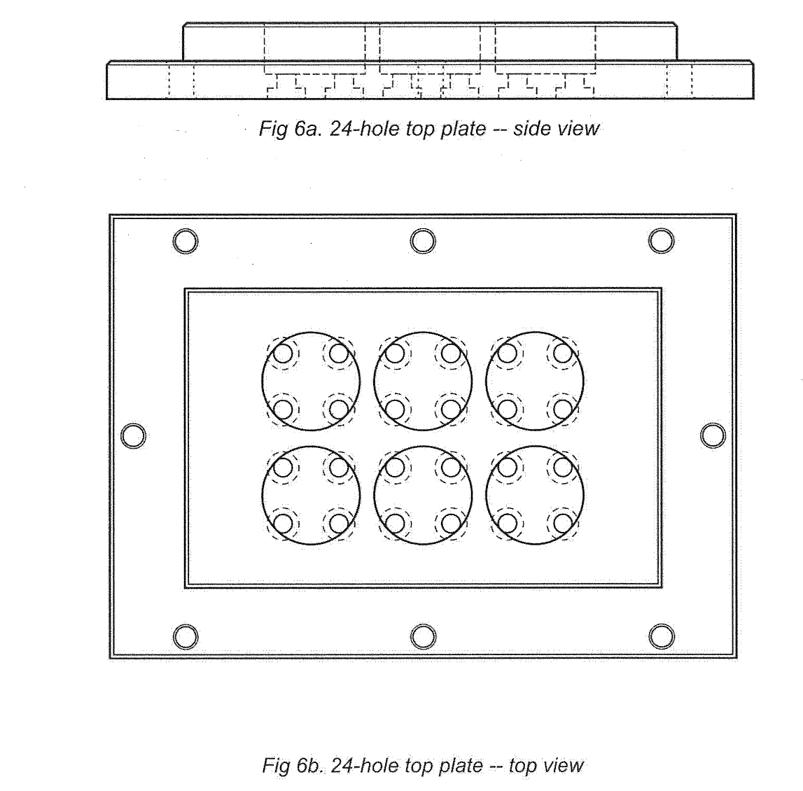

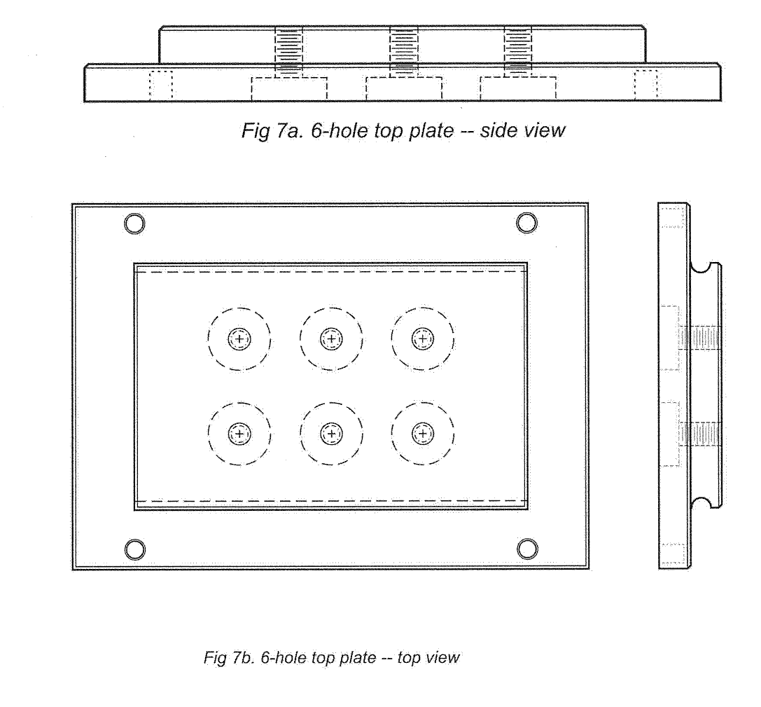

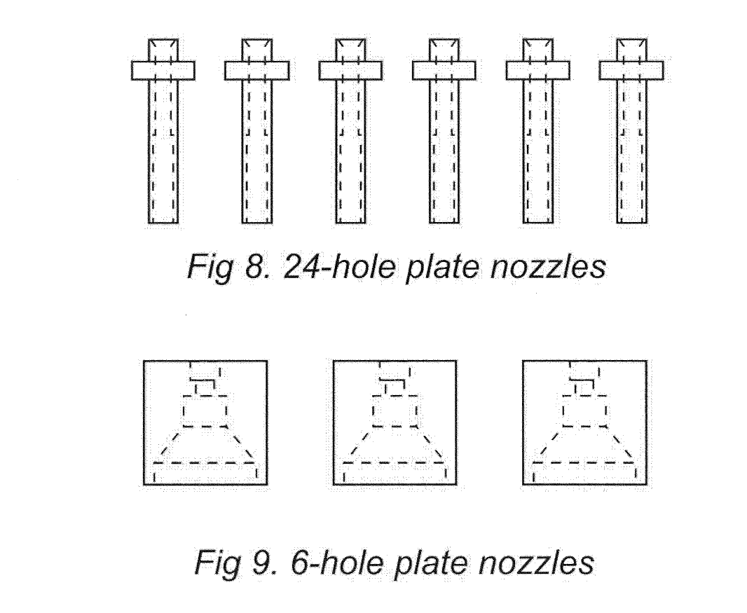

Regarding the exposure method, FIG. 1 presents an exploded view of the design for a cell culture exposure system (CCES) device 100. Aerosols, vapors, or gases (“exposure atmosphere”) 101 enters the CCES through inlet port 102. Exposure atmosphere 101 may comprise, for example, combustion emissions generated and emitted from an exposure atmosphere generation system 120 (e.g., a diesel engine or a simulated-smog generation unit), or ambient air. The exposure atmosphere 101 is delivered at the desired concentration under a slight positive pressure via inlet port 102, which fluidly connects with inlet distribution manifold 103 creating mixing within inlet distribution manifold 103. Inlet distribution manifold 103 separates and reduces the exposure atmosphere air flow rates (and may reduce exposure atmosphere concentration by additional dilution, by supplying additional air via supplemental port 121) to each inlet mixing manifold 104. Mixing manifold 104 increases the mixing and uniformity of the exposure atmosphere for delivery via fluid channels 105 to ALI nozzles 106 (FIGS. 8 & 9). ALI nozzles 106 (FIGS. 8 & 9) are positioned within top plate 113 and extend individually down into each cell culture plate well 107 (shown in FIG. 2A or 2B) within cell culture plate receptacle 111 when the top plate 113 (FIGS. 6A, 6B, 7A, & 7B) is placed down on bottom plate 114 (FIGS. 4A & 4B), to deliver the exposure atmosphere for interaction with the living cells 108 in the ALI inserts 109 (again as shown in FIG. 2A or 2B). Cells 108 (e.g., living murine or human cells) are grown and reside on porous membrane 116, which rests in a culture medium 110 by the ALI inserts 109, as is known in the art. ALI inserts 109 reside in cell culture plate and then placed in the cell culture plate receptacle 111, and may be inserted, removed or replaced in CCES 100 as necessary. In a preferred embodiment, cell culture plates are common, interchangeable cell culture plates that are commercially available from many manufacturers (e.g. Transwell®, Snapwell™, Netwell®, and Falcon®). The number of ALI nozzles 106 (FIGS. 8 & 9) and matching number of ALI inserts 109 may vary but generally 6 (FIG. 9) or 24 (FIG. 8) will be used, depending on the cell culture plates used for the exposure.

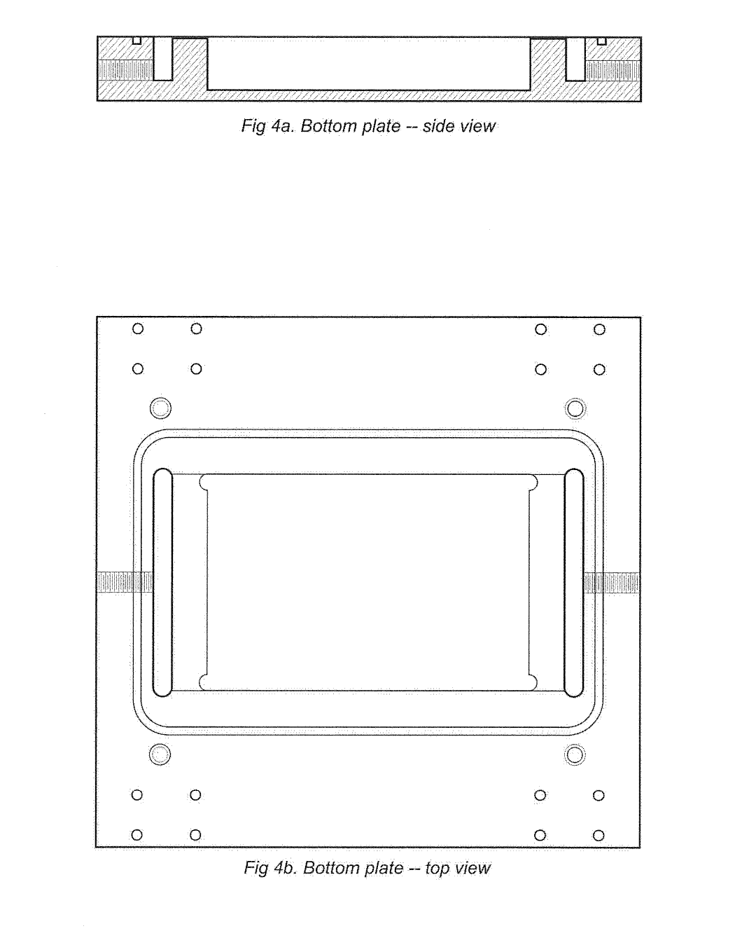

The various cell wells 107 are openly connected with each other in a closed system so that each cell well is maintained under a slight positive pressure. A closed system is created by placing top plate 113 (FIGS. 6A, 6B, 7A, & 7B) on bottom plate 114 (FIGS. 4A & 4B) and then temporarily sealing the top plate 113 (FIGS. 6A, 6B, 7A, & 7B) to bottom plate 114 (FIGS. 4A & 4B) as desired through a closure means 115. In the preferred embodiment shown in FIG. 1, the closure means is shown as multiple hinged clamps 115 that effectively prevent the exposure atmosphere 101 from exiting cell culture plate receptacle 111 except as specified hereafter. Thus, cell culture plate wells 107 remain under a slightly positive pressure to allow for continuous delivery of fresh exposure atmosphere 101. Other closure means (e.g. adhesives, interlocking methods, etc) could also be used. Exhaust manifolds 112a (FIG. 5) and 112b are under a slight negative pressure and pull spent exposure atmosphere from each well 107. All air flow from the exhaust manifolds 112a (FIG. 5) and 112b are exhausted from the system via one or more exhaust ports 118.

FIGS. 2A and 2B show the flow of exposure atmosphere 101 through ALI nozzle 106 into wells 107 for interaction with the living cells 108 within ALI inserts 109. As shown in FIG. 2B, exposure atmosphere 101 may include the flow of particles 117 for testing. Complex mixtures may contain particles 117 and volatile chemicals that are not easily tested with traditional in vitro tools.

When exposing cells to gases or vapors the nozzles 106 (FIG. 8) and ALI inserts 109 are preferably heated to the same 37° C. temperature, and the air flow rates are increased to enhance mixing of the exposure atmosphere. However, when exposing cells to aerosols with suspended particles for testing, the nozzles 106 (FIG. 9) are preferably instead heated to approximately 45° C. (shown as heated nozzles 106b in FIG. 2B). The resulting presence of a temperature gradient (AT) (shown in FIG. 2B at 119) between the nozzles 106b and plates 109 imposes a thermophoretic force on the particle environment for particle transfer to the inserted plate 109. In addition, airflow may also be reduced to provide higher efficiency for aerosol 101 delivery to the cells 108. When more complex mixtures (aerosols and vapors) are used for exposure atmosphere 101, the best airflow rate is chosen to maximize exposure to the complex mixture or individual components (aerosols or vapors) and to maximize differentiation of the biological effects.

Preferably, when thermophoresis is used the temperature gradient is no more than 10 degrees Celsius (i.e., 37° C. at plates 109 and 47° C. at nozzles 106b) as otherwise the heat transfer to the cells becomes too great and cell death may increase.

### Example 2

In order to compare human cells response as compared to a standard cell culture, the following test was performed:

Human primary lung cells and BEAS-2B cells were evaluated by exposure to 1,3-butadiene. 1,3-Butadiene is a combustion emission found in ambient air in urban and suburban areas as a result of its emission from motor vehicles. The EPA lists it as the “mobile-source air toxic” with the highest normalized risk factor, exceeding that of formaldehyde, the second riskiest air toxic emitted by motor vehicles, by a factor of more than 20. 1,3-butadiene is an IARC Group 1 known human carcinogen. Cells were exposed at the air-liquid interface (ALI) using the EPA's Cell Culture Exposure System (CCES) as described above. Levels of cytotoxicity were measured post-exposure, and RNA samples were collected and shipped to BioSpyder to measure gene expression.

### Methods

### Exposure System

The Cell Culture Exposure System (CCES) provides the ability to expose cultured mammalian cells to airborne chemicals at ALI conditions in a 24-well format.

### Human Primary Lung Cells

Human primary lung cells were collected by cytology brush biopsy during bronchoscopy at EPA clinical facility in Chapel Hill. Cells were cultured by standard procedures and were provided to EPA/RTP.

### BEAS-2B Cells

The BEAS-2B cell line, obtained from ATTC, is an immortalized human bronchial epithelial lung cell.

### Culturing on Transwell Membranes

### BEAS-2B Cells:

Cells were grown out on T75 cm² flasks in complete Keratinocyte Growth Media (KGM) (KGM-Gold Bullet Kit, Lonza®). Cells were passaged and seeded onto 6.5 mm (24-well format) permeable (0.4-μm pore size) Transwell polyester membranes at passages 55 and 56. Prior to seeding, Transwells were coated with PureCol® bovine collagen (Advanced Biometrix) at a density of 10 μg/cm². Cells were seeded at 3.0×10⁴ cells per Transwell membranes 48 h prior to exposure and at 1.5×10⁴ per Transwell membrane 72 h prior to exposure to obtain a confluent monolayer. Cells were seeded on 4 inserts per exposure condition for each run.

### Human Primary Lung Cells:

Cells were cultured and plated onto Transwell membranes and grown out for 28 days as described in OP.

### Exposure Conditions

Cells were placed into new 24-well plates with fresh growth media and put at ALI 2 h prior to exposure. Cells were exposed to 6 different concentrations of 1,3-butadiene for 2 h using the CCES. Target concentrations were 50, 16, 5, 1.5, 0.5, and 0.16 ppm 1,3-butadiene. Control cells were exposed simultaneously to clean air for 2 h in a separate chamber. Another set of control cells were kept at ALI in an incubator (37° C., 5% CO₂) for 2 h. After the exposure, cells were placed in an incubator (37° C., 5% CO₂) for 4 h. A total of three exposures were conducted for each cell type.

### 1,3-Butadiene Source

The 1,3-butadiene was 1000 ppm in a balance of air in a compressed gas cylinder purchased from a commercial source that was diluted into the desired concentrations.

### Viability Assay

The CellTiter-Glo® Luminescent Cell Viability Assay (Promega) determines the number of viable cells based on quantification of the ATP present; an indicator of metabolically active cells. Analysis was conducted on cell samples 4 h post-exposure to quantify the viability resulting from exposures to 1,3-butadiene. Two inserts per exposure condition were measured for each run.

### Cytotoxicity Assay

The Pierce® LDH cytotoxicity assay measures cell cytotoxicity though the quantification of lactate dehydrogenase (LDH). Plasma membrane damage releases the LDH into the cell culture media. Analysis was conducted on cell samples 4 h post-exposure to quantify the viability resulting from exposures to 1,3-butadiene. Two inserts per exposure condition were measured for each run.

### Gene Expression

Cells were lysed using BioSpyder TempO-Seq lysis buffer as described by the manufacturer and provided to Josh Harrell for shipment to BioSpyder Corporation for analysis of expression of the ˜20,000 human coding genes. Results

### 1.) Human Primary Lung Cell Results

The CellTiter-Glo results for the human primary lung cells for all 3 experiments are shown below both in the table and graph. No significant reduction in viability was observed at any dose of 1,3-butadiene. There was no significant difference in viability between the incubator control and the clean air control. Data from the LDH cytotoxicity assay support the data from CellTiter-Glo.

TABLE 1
CellTiter-Glow viability for human primary lung cell
exposures to 1,3-Butadiene. Human Primary Lung Cells

| | May 3, | | | | |
|---|---|---|---|---|---|
| | 2017 | May 4, 2017 | May 5, 2017 | Average | STDEV |
| 50 ppm | 104.7 | 92.7 | 95.4 | 97.6 | 6.2730248 |
| 16 ppm | 128.1 | 101.3 | 98.9 | 109.4 | 16.193108 |
| 5 ppm | 122.0 | 112.0 | 98.6 | 110.8 | 11.754136 |
| 1.7 ppm | 120.3 | 91.8 | 101.4 | 104.5 | 14.457869 |
| 0.5 ppm | 118.5 | 86.3 | 94.2 | 99.6 | 16.797697 |
| 0.16 ppm | 110.9 | 99.9 | 90.1 | 100.3 | 10.431488 |
| Clean Air | 106.1 | 95.5 | 93.7 | 98.4 | 6.7390373 |
| Inc. Ct. | 100.0 | 100.0 | 100.0 | 100.0 | 0 |

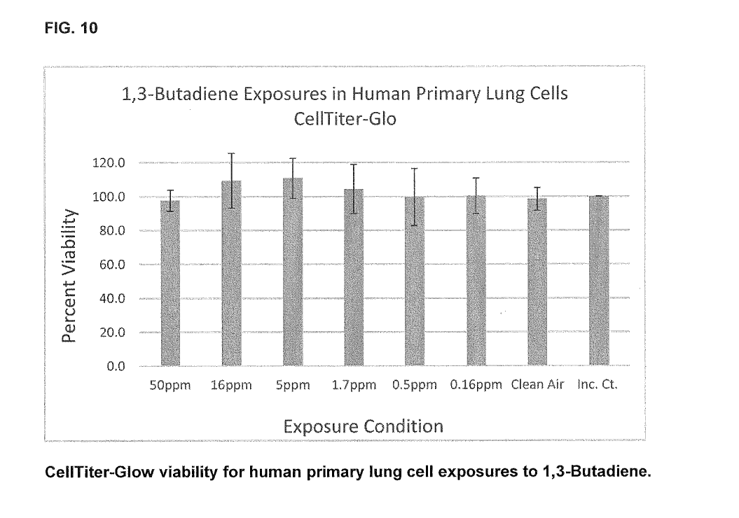

### See FIG. 10 Regarding Exposure Conditions,

### 2.) BEAS-2B Results

Below is a table and figure of the CellTiter-Glo results from all 3 experiments involving the exposure of BEAS-2B cells. Again, there was no significant difference in viability between the clean air control and the incubator control. Using data from all 3 experiments, p=0.21, and from just the last two experiments, p=0.58. We considered the analysis using data from only the last 2 experiments because in the first experiment, the greatest amount of cell killing occurred in the clean air control, indicating an error in the system. Therefore, comparisons of viability among exposed cells were made to the clean air control. Using data from all 3 experiments, the 50 ppm versus clean air control had a p=0.35, indicating that at the highest dose of 1,3-butadiene there was no significant decrease in viability. Using data from only the last 2 experiments, the p=0.22, also indicating no significant reduction in viability at the highest dose (50 ppm). Data from the LDH cytotoxicity assay support the data from CellTiter-Glo.

TABLE 2
CellTiter-Glow Viability for BEAS-2B exposures to 1,3-Butadiene.
BEAS-2B Cells

| | May 10, 2017 | May 11, 2017 | May 12, 2017 | Average | STDEV |
|---|---|---|---|---|---|
| 50 ppm | 105.1339519 | 71.10453129 | 9.719941515 | 61.9861416 | 48.356149 |
| 16 ppm | 101.5423355 | 87.3133717 | 79.13917364 | 89.3316269 | 11.337126 |
| 5 ppm | 105.2864922 | 81.90200149 | 96.79729291 | 94.6619289 | 11.837586 |
| 1.7 ppm | 94.35344245 | 94.49344987 | 95.83375519 | 94.8935492 | 0.817246 |
| 0.5 ppm | 101.7881978 | 89.2982651 | 96.0984404 | 95.7283011 | 6.2531878 |
| 0.16 ppm | 96.26697349 | 87.13821567 | 96.95102568 | 93.4520716 | 5.4786462 |
| Clean | 83.68623228 | 89.82831516 | 102.2542116 | 91.9229197 | 9.4595449 |
| Air | | | | | |
| Inc. Ct. | 100 | 100 | 100 | 100 | 0 |

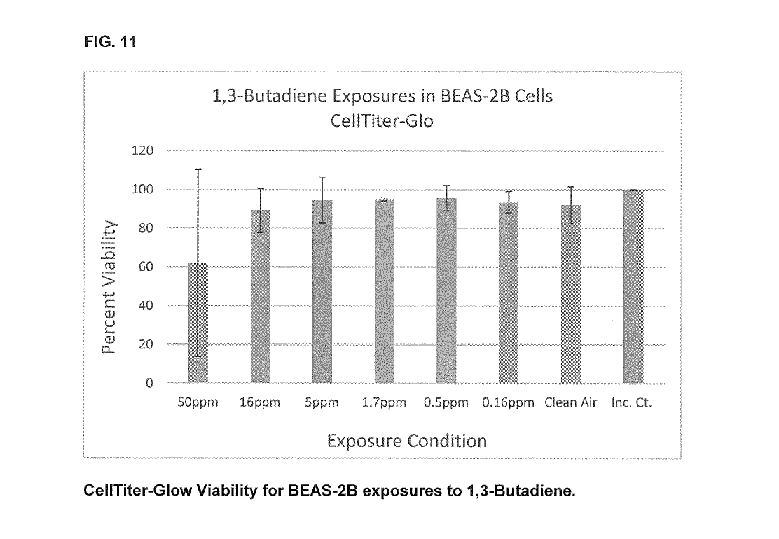

### See FIG. 11 Regarding Exposure Conditions.

There are many benefits of the CCES 100 as described above. These include (1) inherent multiplicity of design for expansion to multiple cell culture plates with minimal adaptation of the exposure system, (2) the ability to tailor efficiency of the test article (i.e. gas or particle modes) being delivered with exposure of cell cultures, (3) the cost and convenience benefit of being able to use standard commercially available cell culture plates, (4) simplicity and ease of design, and (5) use of thermophoresis to enhance particle deposition.

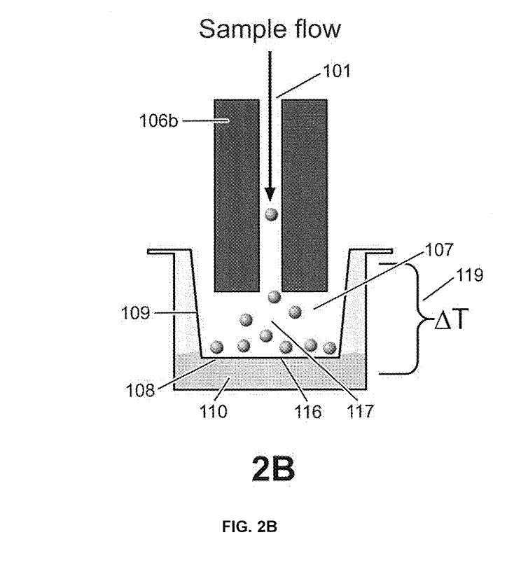

An alternative embodiment of the invention is illustrated in FIG. 3. As illustrated in FIG. 3, a widely-utilized nose-only in-vivo exposure system may also be accommodated in the CCES, and therefore one exposure system may be used for both in-vitro and in-vivo exposures. This may be done simultaneously, and may be maximized for either in-vitro or in-vivo exposure scenarios. As shown in FIG. 3, alternative CCES 200 comprises exposure atmosphere generation system 201 providing exposure atmosphere 202 for evaluation. Exposure atmosphere 202 may optionally pass through humidifier 203 prior to being provided to the cell culture or animal specimens. Nose-only exposure chamber 204 receives the exposure atmosphere 202 and distributes the exposure atmosphere 202 to animal specimens 211 in nose-only exposure tubes 210 for in vivo testing, as well as distributing the exposure atmosphere 202 to the CCES 206 (via inlet mixing manifolds 205 and ALI nozzles 214) for simultaneous in vitro testing on CCES 206 in the same manner as described for the preferred embodiments above. On the in vitro side of CCES 200, a vacuum pump 208 and mass flow controller(ds) (209) are used to control air flow through each CCES on the system. Spent exposure atmosphere is again exhausted through an exhaust port (not shown). On the in vivo side of CCES 200, HEPA filter 213 is preferably provided prior to exhaust of the exposure atmosphere from the system. The CCES 200 and nose-only exposure chamber 210 are both operated as “push-pull” systems with the differential static pressure of both systems being slightly positive.

As can be seen, this alternative embodiment 200 facilitates better comparison of exposure results between animal models for specific diseases and cell culture assays for specific biological endpoints. The CCES 200 in FIG. 3 enables exposure of either or both to the same exposure atmosphere generated from the same source under the same conditions, and therefore minimizes the variables that are changed between the two exposure regimens. This approach therefore provides important data for understanding human diseases or health outcomes from specific exposure scenarios.

What we claim is:

1. A method of evaluating effect of a polluting air stream comprising the steps of:
a) exposing cells to a membrane until adhesion of the cells has occurred,
b) feeding cells periodically until there is a confluent monolayer of cells on said membranes,
c) aspirating off non-adherent cells
d) applying fresh media
e) exposing the cells to over-head stream containing pollutants
f) removing the cells from the membranes that have been exposed to the over-head stream, then
g) measuring the cells' response to toxins in the over-head stream.

2. The method of claim 2 wherein the membranes have been coated with collagen.

3. The method of claim 1 wherein the cells used are from a commercially available cell line.

4. The method of claim 2 wherein the cells are lung cells.

5. An apparatus for use in evaluating effect of pollution on cells comprising an inlet port for admission of the exposure atmosphere from an atmosphere generator system comprising an inlet port which fluidly connects with an inlet distribution manifold to separate and control stream containing exposure atmosphere, a mixing manifold, a series of fluid delivery channels leading to nozzles position in a plate, said nozzles being positioned within a plate, said nozzles extending down into a cell culture plate containing living.

6. The apparatus of claim 5 wherein the contain living cells on a porous membrane.

7. The apparatus of claim 6 wherein the porous membrane is coated with collagen.
